(19) United States
(12) Patent Application Publication
(10) Pub. No.: US 2002/0090379 A1
(43) Pub. Date: Jul. 11, 2002

(54) INDUCING ANTIBODY RESPONSE AGAINST SELF-PROTEINS WITH THE AID OF FOREIGN T-CELL EPITOPES

(76) Inventors: SOREN MOURITSEN, BIRKEROD (DK); HENRIK ELSNER, BRONSHOJ (DK)

Correspondence Address: JACOBSON PRICE HOLMAN AND STERN, THE JENIFER BUILDING, 400 SEVENTH STREET NW, WASHINGTON, DC, 200042201

(21) Appl. No.: 08/955,373

(22) Filed: Oct. 21, 1997

Related U.S. Application Data
(63) Continuation of application No. 08/803,321, filed on Feb. 21, 1997, now abandoned.
Application No. 08/803,321 is a continuation of application No. 08/477,501, filed on Jun. 7, 1995, now abandoned.
Application No. 08/477,501 is a continuation-in-part of application No. PCT/DK94/00318, filed on Aug. 25, 1994.

(30) Foreign Application Priority Data
Aug. 26, 1993 (DK) 0964/93

(51) Int. Cl. A61K 39/00; A61K 39/38; A61K 39/385; C07K 1/00; C07K 14/00; C07K 17/00; G01N 33/53

(52) U.S. Cl. 424/185.1; 424/192.1; 424/193.1; 424/198.1; 530/350; 436/547; 436/548

(57) ABSTRACT

A novel method for utilizing the immune apparatus to remove and/or down-regulate self-proteins. The method consists in providing a self-protein analog by molecular biological means by substitution of one or more peptide fragments of the self-protein by corresponding number of peptides known to contain immunodominant foreign T-cell epitopes, said substitution being carried out so as to essentially preserve the overall tertiary structure of the original self-protein. This render the self-protein immunogenic and leads to a rapid induction of high-titered autoantibodies against the native self-proteins. The modulated self-proteins can be used to prepare vaccines against undesirable proteins in humans or animals, said vaccine being useful as therapeutics against a number of diseases, e.g. cancer, chronic inflammatory diseases such as rheumatoid arthritis and inflammatory bowel diseases, allergic symptoms or diabetes mellitus.

### CROSS REFERENCE TO RELATED APPLICATION

The present application is a continuation-in-part of international application PCT/DK94/00318 filed on Aug. 25, 1994.

### BACKGROUND OF THE INVENTION

Physiologically, the vertebrate immune system serves as a defence mechanism against invasion of the body by infectious objects such as microorganisms. Foreign proteins are effectively removed via the reticuloendothelial system by highly specific circulating antibodies, and viruses and bacteria are attacked by a complex battery of cellular and humoral mechanisms including, antibodies, cytotoxic T lymphocytes, Natural Killer cells, complement etc. The leader of this battle is the T helper (T_{H}) lymphocyte which, in collaboration with the Antigen Presenting Cells (APC), regulate the immune defence via a complex network of cytokines.

T_{H} lymphocytes recognize protein antigens presented on the surface of the APC. They do not recognize, however, native antigen per se. Instead, they appear to recognize a complex ligand consisting of two components, a “processed” (fragmented) protein antigen (the so-called T cell epitope and a Major Histocompatibility Complex class II molecule (O. Werdelin et al. Imm. Rev. 106, 181 (!088)). This recognition eventually enables the T_{H} lymphocyte specifically to help B lymphocytes to produce specific antibodies towards the intact protein antigen (Werdelin et al., supra). A given T cell only recognize a certain antigen-MHC combination and will not recognize the same or another antigen presented by a gene product of another MHC allele. This phenomenon is called MHC restriction.

Fragments of self-proteins are also presented by the APC, but normally such fragments are ignored or not recognized by the T helper lymphocytes. This is the main reason why individuals generally do not harbour autoantibodies in their serum eventually leading to an attack on the individual's own proteins (the so-called self- or autoproteins). However, in rare cases the process goes wrong, and the immune system turns towards the individual's own components, which may lead to an autoimmune disease.

The presence of some self-proteins is inexpedient in situations where they, in elevated levels, induce disease symptoms. High levels of immunoglobulins of the IgE class are e.g. known to be important for the induction of type I allergy, and tumor necrosis factor α (TNFα) is known to be able to cause cachexia in cancer patients and patients suffering from other chronic diseases (H. N. Langstein et al. Cancer Res. 51, 2302-2306, 1991). TNFα also plays important roles in the inflammatory process (W. P. Arend et al. Arthritis Rheum. 33, 305-315, 1990). Hormones in sex-hormone dependent cancer are other examples of proteins which are unwanted in certain situations. There is therefore a need for the provision of a method for the development of vaccines against such self-proteins.

1. Field of the Invention

This invention concerns a novel method for utilizing, the immune apparatus to remove and/or down-regulate self-proteins, the presence of which somehow is unwanted in the individual because such proteins are causing disease and/or other undesirable symptoms or signs of disease. Such proteins are removed by circulating autoantibodies which specifically are induced by vaccination. This invention describes a method for developing such autovaccines.

2. Description of the Prior Art

It is possible artificially to induce antibodies against self-proteins. This can be done, by covalent conjugation of the self-protein, or appropriate synthetic peptides derived from the self protein. To an appropriate large foreign carrier protein as e.g. tetanus toxoid or key-hole limpet haemocyanin (KLH). Talwar et al. (G. P. Talwar et al, Int. J. Immunopharmnacol. 14, 511-514, 1992) have been able to prevent reproduction in women using a vaccine consisting of a conjugate of human chorionic gonadotropin and tetanus toxoid. There are also other examples of such autoimmunogenic conjugates which have been used therapeutically in man and in animal models (D. R. Stanworth et al. 336, 1279-1281, 1990). During the processing of such conjugates in the APC, the necessary T_{H} lymphocyte stimulatory epitopes are provided from the foreign protein eventually leading to the induction of antibodies against the carrier protein as well as against the self-protein. One disadvantage of using this principle is, however, that the antibody response towards the self-protein will be limited due to shielding of epitopes by the covalently linked carrier protein. Another disadvantage is the increased risk of inducing allergic side-effects due to the contemporary induction of an undesired very strong antibody response against the foreign carrier protein.

Other researchers have conjugated a single peptide predicted to be a T cell epitopes chemically as carrier to a self-peptide [D-Lys⁶] GnRH, which is a decapeptide acting as a hapten, and managed to induce an autoantibody response with MHC restriction to that particular T cell epitope (S. Sad et al., Immunology 76, 599-603, 1992). This method seems to be more effective compared with conjugation to large carrier proteins. However, it will only induce antibodies in a population expressing the appropriate MHC molecules. This means that a rather large number of different T cell epitopes has to be conjugated to the self-peptide which eventually would disturb the B-cell epitopes on the surface of the self-peptide. Extensive conjugation of proteins may furthermore have the opposite effect with regard to immunogenicity (WO Ser. No. 87/00056) and the surface exposed peptide T cell epitopes may be destroyed by proteolytic enzymes during antigen processing (S. Mouritsen, Scand. J. Immunol, 30, 723, 1989 makings this method inexpedient. Also the exact structure of such multi-conjugated self-peptides will not be chemically and pharmaceutically well defined.

The method described in WO Ser. No. 92/19746 involves vaccines against LHRH which is a self-peptide (not a self-protein) consisting of 10 amino acids. The vaccines may be produced by recombinant methods and involve constructs comprising the intact native peptide fused to one or more known T-cell epitopes and a purification sit. Thus, this document is not concerned with self-proteins in which one or more peptide fragments have been replaced with foreign T-cell epitopes, but with a self-decapeptide having an intact primary structure and obviously the tertiary structure is not essentially preserved. Apart from the fact that it rarely has meaning to consider the tertiary structure to a peptide, the fusion of a peptide to a T-cell epitope of at least the sate size or even larger would most probably distort any such tertiary structure. From the observations made in WO Ser. No. 92/19746 it is not obvious that T-cell epitopes inserted into an autologous protein by the method of the present invention would elicit such a strong and rapid immune response as observed. The surprising observations underlying the present invention are a consequence of the fact that the T-cell epitopes are inserted into the self-protein, against which it is the purpose to raise antibodies. The epitopes substitute the self-protein fragments, thus preserving the overall secondary and tertiary structure of the self-protein to a large extent. The tolerance towards the self-protein is broken by two supplementary means: By the introduction of a foreign known immunodominant T-cell epitope, which due to its intrinsic immunodominant properties also will be immunodominant in the self-protein, and thus able to provide T-cell help to self reactive B-cells. Secondly, the tolerance is broken by the simultaneous removal of potential self immunodominant self-epitopes to which the organism is tolerized, hereby disturbing the intramolecular competition of epitope binding to MHC class II molecules. This disturbance may, expose cryptic self-epitopes not previously presented to the immune system. A fusion between a T-cell epitope and a self-peptide, as suggested in WO Ser. No. 92/119746 could only give a limited range of different antibodies binding to the decapeptide due to epitope shielding by the fused T-cell epitope, and obviously no potential immunodominant self epitopes could be removed as such an epitope would constitute the complete peptide. It is of importance to essentially preserve the tertiary structures, as it is done in the present invention, because these structures determine the specific recognition of the non-modified self-protein by the induced antibodies. Additionally, the neighbouring regions adjacent to the inserted immunodominant T-cell epitopes in the present invention provide two new border regions between inserted epitope and self-protein sequence, which also contribute to the immunogenicity of the construct This is reflected in the examples of the present application showing an apparent change of the expected MHC restriction pattern in the self-protein analogs with different T-cells epitopes inserted.

The breaking of the autotolerance towards a polypeptide part of a self-protein has also previously been reported in WO Ser. No. 93/05810: “Vaccine comprising part of constant region of IgE for treatment of IgE-mediated allergic reactions”. Here the self polypeptide is chemically coupled to a carrier protein, although it is also suggested to use molecular biological means. A purification “tag” is suggested as the carrier for the ease of purification. In this document no considerations are made regarding the importance of inserting strong well-defined immudominant T-cell epitopes. Single amino acid mutations in the autologous polypeptide are considered and it is speculated, that new T-cell epitopes by chance could emerge from such mutations. In this document a polypeptide fragment, the constant CH₂-CH₃, domains of the much larger IgE protein, are used for raising the autoantibodies and some foreign carrier protein is coupled to this. Thus no considerations are made regarding the importance of essentially preserving the tertiary structure, let alone using the complete protein for facilitating the broadest possible self epitope sequences. Furthermore, the induction of autoantibodies by coupling of the autoantigen to a large carrier protein is not as efficient as the method according to the present invention. By inserting, known immunodominant T-cell epitopes derived from e.g. tetanus toxoid, as suggested in the present invention, an additional important technical advantage is the ability to test in vitro whether the inserted epitopes are correctly processed by the antigen presenting cells and subsequently presented to human tetanus toxoid specific T-cells. This makes it possible to test the immunogenicity of the self-protein analogs without prior immunization of humans with these constructs.

Yet another method has been proposed for breaking the B-cell autotolerance by chemical conjugation of B- and optionally also peptide T cell epitopes to a high molecular weight dextran molecule (WO Ser. No. 93/23076). The disadvantages mentioned above, however, also holds true for this invention, which anyway is clearly different from the herein described method.

The object of the above-mentioned citations are analogous to the intention of the present invention, viz. to raise autoantibodies, but the strategy and scientific considerations are verv different. Consequently, the present applicants have observed a much broader applicability and a stronger and faster attainment of the purpose of raising, autoantibodies using the strategy of the present invention.

It has been suggested previously that a universally recognized strong T cell epitope could be associated with foreign peptide having an antigenic structure representing a B-cell epitope using, recombinant DNA technology (EP-A2-0-343 460). It has also been suggested to use pepticyl resin conjugates comprising an immunogenic or antigenic peptide incorporating a helper T-cell (T_{H}-lymphocyte) reactive epitope and preferably a B-cell reactive epitope, in the preparation of immunogenic compositions, eg. vaccines. The conjugates are prepared by solid phase synthesis preferably on a polyamide resin, (WO Ser. No. 90/15627). While the intent is to increase an antibody response towards the peptides in question, it has not been proposed that it could be done with the purpose of breaking the autotolerance of the immune system, and induce an antibody response against self-proteins.

### SUMMARY OF THE INVENTION

The problem to be solved by the present invention is to provide immunogenic compositions which are capable of inducing a high-titered and rapid antibody response in a heterogeneous MHC-population against pathogenic self-proteins, so that vaccines against said proteins can be prepared.

The solution to this problem is based on the surprising finding that the substitution by molecular biological means of one or more peptide fragments in a self-protein by a corresponding number of immunodominant foreign T-cell epitopes in such a way that the tertiary structure of the self-protein is essentially preserved render this self-protein analog highly immunogenic leading to a profound antibody response against the unmodified self-protein. By substituting the epitopes into the self-protein the immune response elicited is furthermore not only restricted to the known MHC class II type of the inserted immunodominant T-cell epitope, but the modified self-protein also elicits an autoantibody response in other MHC-haplotypes. Consequently, the recombinant self-protein analogs will be self-immunogenic in a large population expressing many different MHC class II molecules.

Using this methodology it was possible to induce strong antibodies against e.g. the highly conserved self-protein, ubiquitin, as well as the inflammatory cytokine, TNFα.

### BRIEF DESCRIPTIONS OF THE DRAWINGS

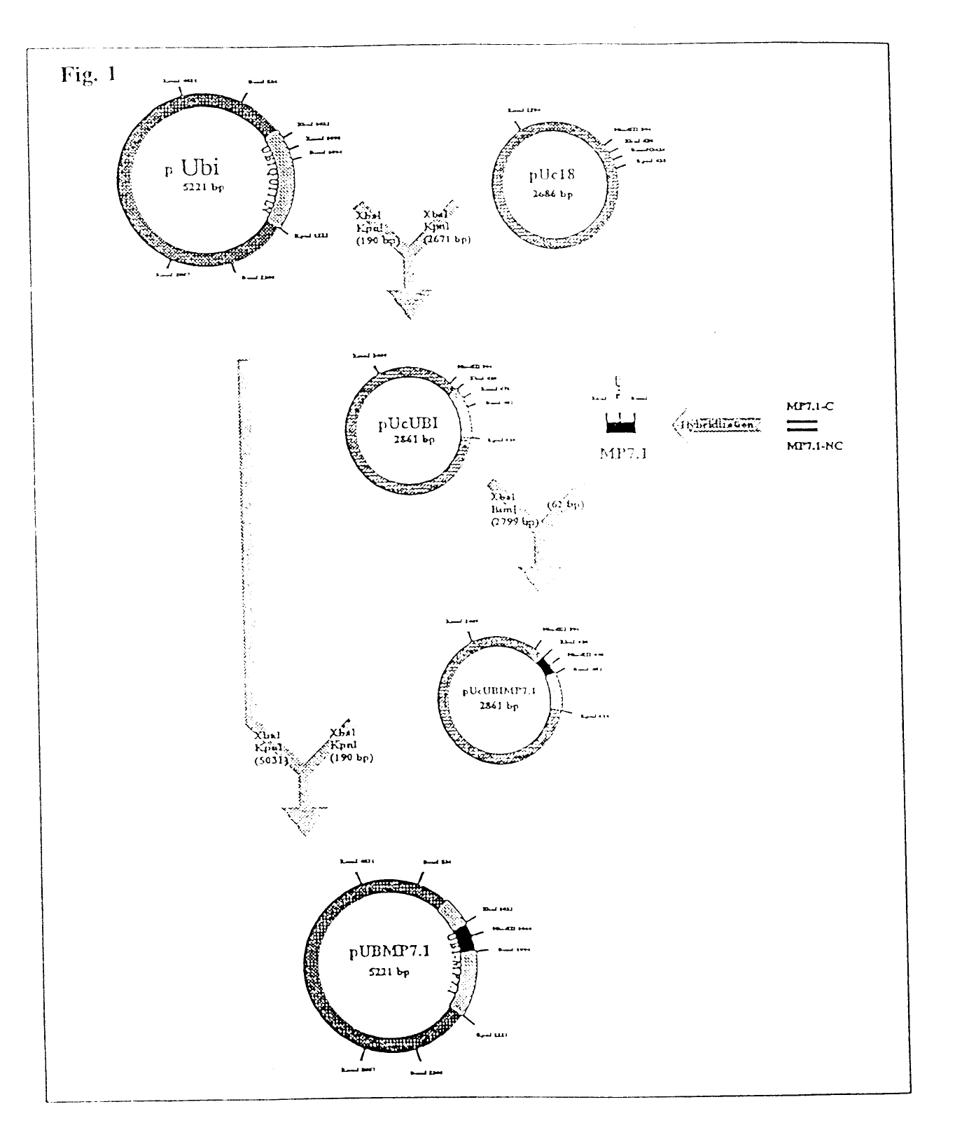

### Legend to FIG. 1

Schematic overview of the cloning strategy used in the construction of a ubiquitin gene with an implanted foreign T cell epitope (MP7). Restriction enzyme digestions, hybridization and ligation procedures are indicated with arrows. Fragment sizes are shown in parentheses.

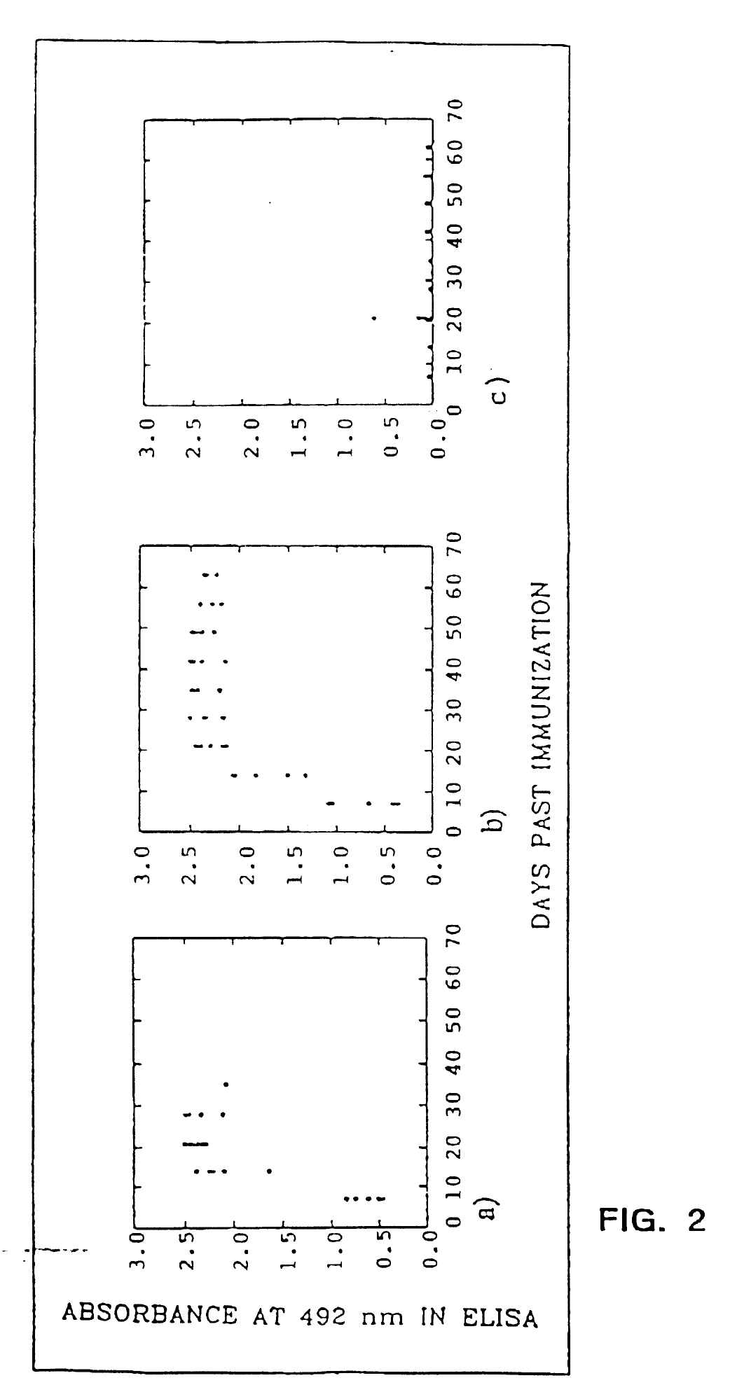

### Legend to FIG. 2

Reactivity towards immobilized bovine ubiquitin in sera from mice immunized with recombinant ubiquitin and analogs containing the implanted T cell epitopes OVA(325-336) and HEL(50-61), respectively. FIG. 2a) sera from Balb/c mice immunized with recombinant ubiquitin containing OVA(325-336). FIG. 2b) sera from Balb/c mice immunized with recombinant ubiquitin containing the T cell epitope HEL(50-61). FIG. 2c) sera from Balb/c mice immunized with recombinant non-modified ubiquitin. Sera (diluted 1:100) were tested in a standard ELISA assay using non-modified bovine ubiquitin immobilized on the solid phase.

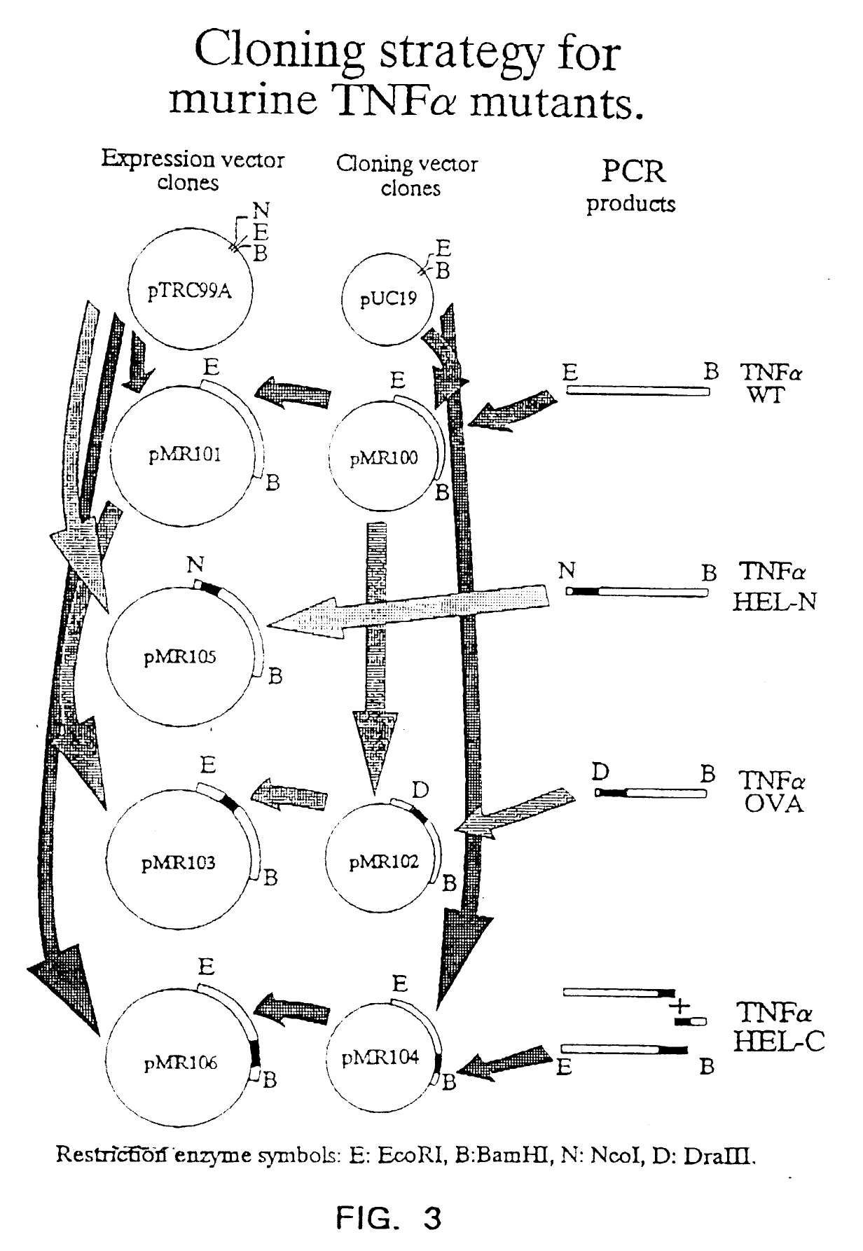

### Legend to FIG. 3

Schematic overview of the cloning strategy used in the construction of the recombinant TNTα mutants. PCR products and restriction enzyme digestions are indicated.

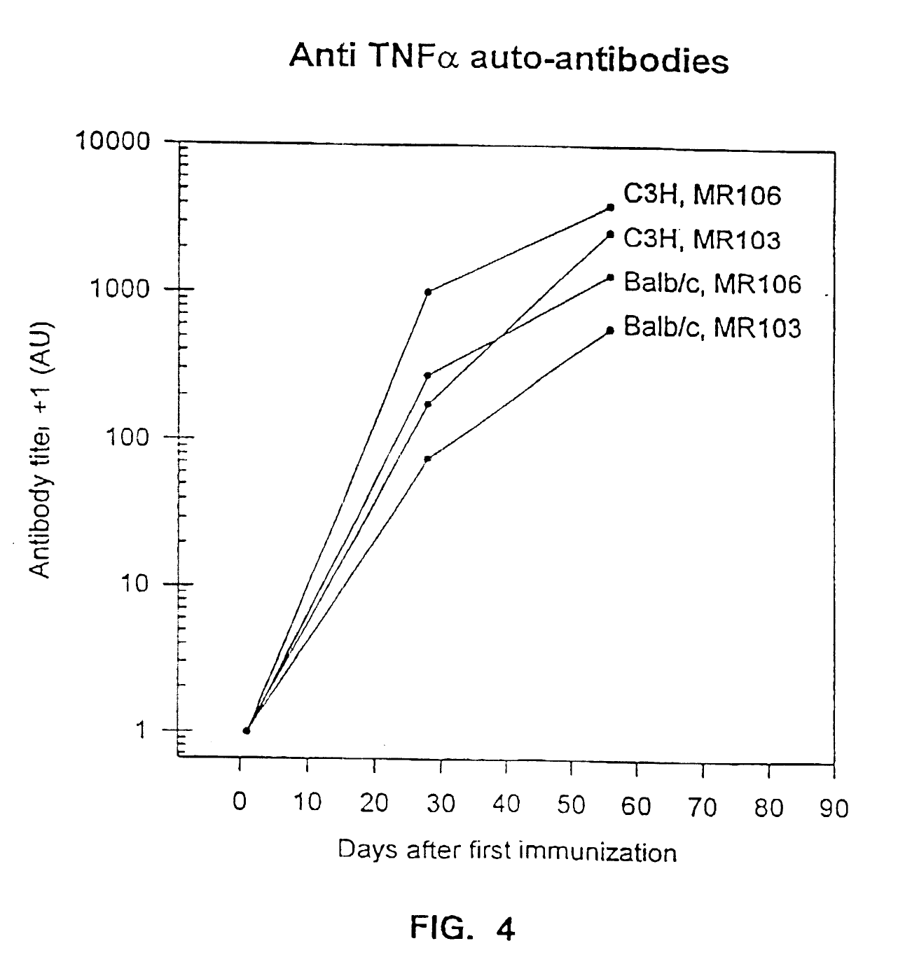

### Legend to FIG. 4

Induction of TNFα autoantibodies by vaccination of Balb/c or C3H mice with the TNFα analogs, MR103 and MR106, respectively. The antibody titers were measured by ELISA and expressed as arbitrary units (AU) referring to a strong standard anti-serum from one mouse. The plotted values represents a mean titer for 5 animals. Freunds complete adjuvant was used as adjuvant for the first immunization. All subsequent immunizations at 14 days intervals were done with Freunds incomplete adjuvant. Mice immunized in parallel with native MR101, or PBS did not develop detectable TNFα autoantibodies (data not shown). Non-detectable antibody titers were assigned the titer value 1.

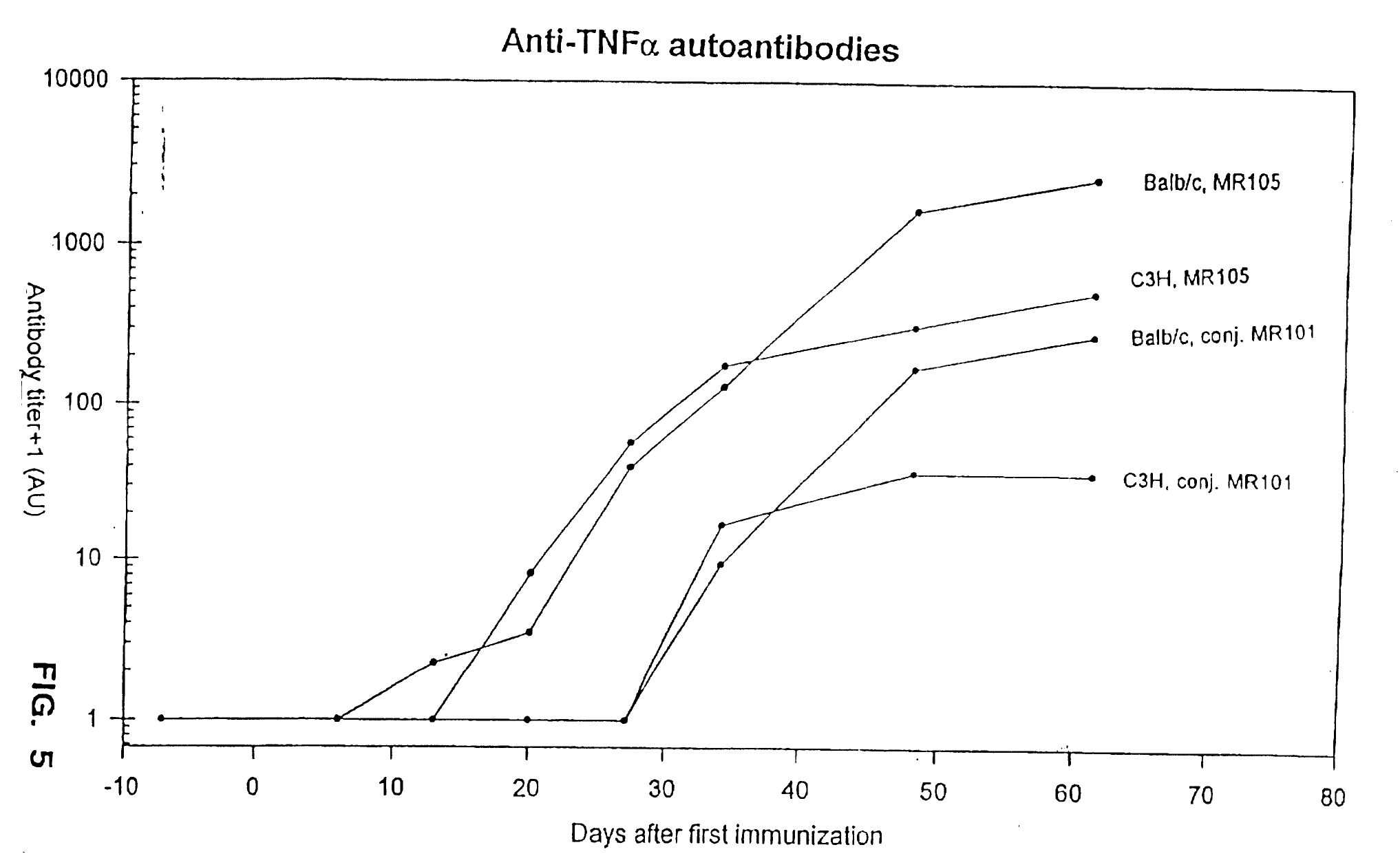

### Legend to FIG. 5

Recombinant murine TNF (MR101) was conjugated to E. coli proteins in PBS, pH 7.4 using 0.5% formaldehyde. Conjugation of the proteins was confirmed by SDS-PAGE. These conjugates were subsequently used for vaccination of the mice. Another group of mice was vaccinated with semipurified non-conjugated self protein analog MR105. About 100 μg of recombinant TNF(αanalog and conjugate were emulsified in Freunds complete adjuvant were injected subcutaneously in each group of mice. In subsequent immunizations every second week, incomplete Freunds adjuvant was used.

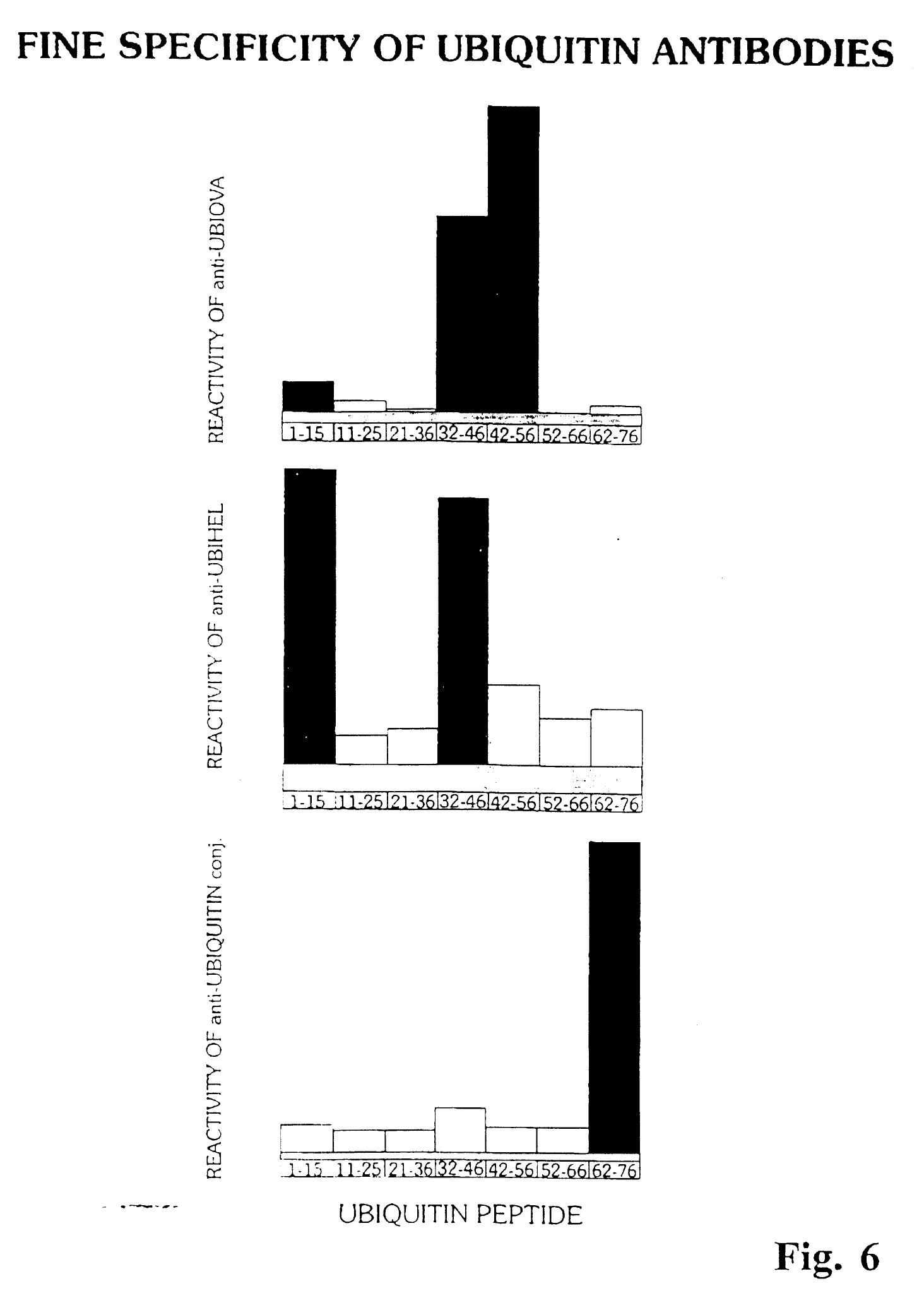

### Legend to FIG. 6

Reactivity against immobilized overlapping ubiquitin peptides in sera from mice immunized with recombinant ubiquitin analogs as well as in serum from rabbits immunized with carrier-coupled bovine ubiquitin. FIG. 6a) serum from Balb/c mice immunized with recombinant ubiquitin containing OVA(325-336). FIG. 6b) serum from Balb/c mice immunized with recombinant ubiquitin containing HEL(50-61). FIG. 6c) serum from rabbits immunized with bovine ubiquitin chemically coupled with human IgG. Pooled sera (diluted 1:50) were tested in an ELISA assay with overlapping synthetic ubiquitin peptides immobilized on activated polystyrene plates.

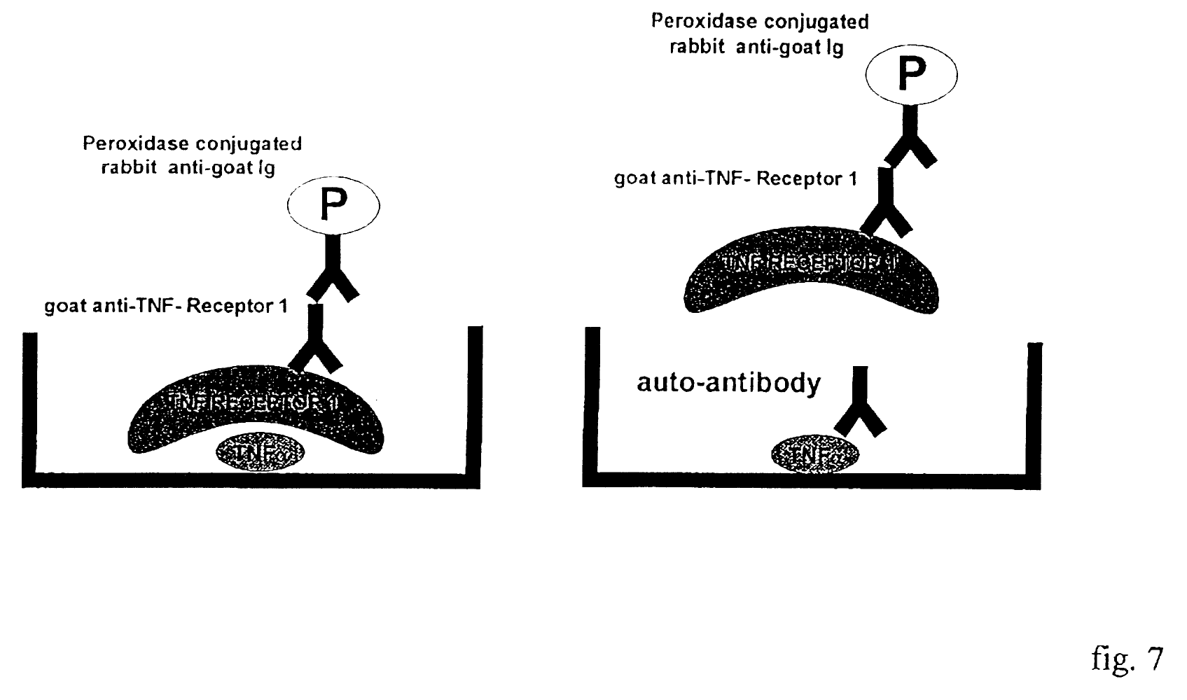

### Legend to FIG. 7

The principle of the ELISA assay used for quantification blocking antibodies. TNFα immobilised on microtiter plates was preincubated for 1 hour with serum from MR106 vaccinated mice. TNR-R1 was added. After repeated washing, bound TNR-R1 was measured by using a peroxidase conjugated goat anti TNR-R1 antibody.

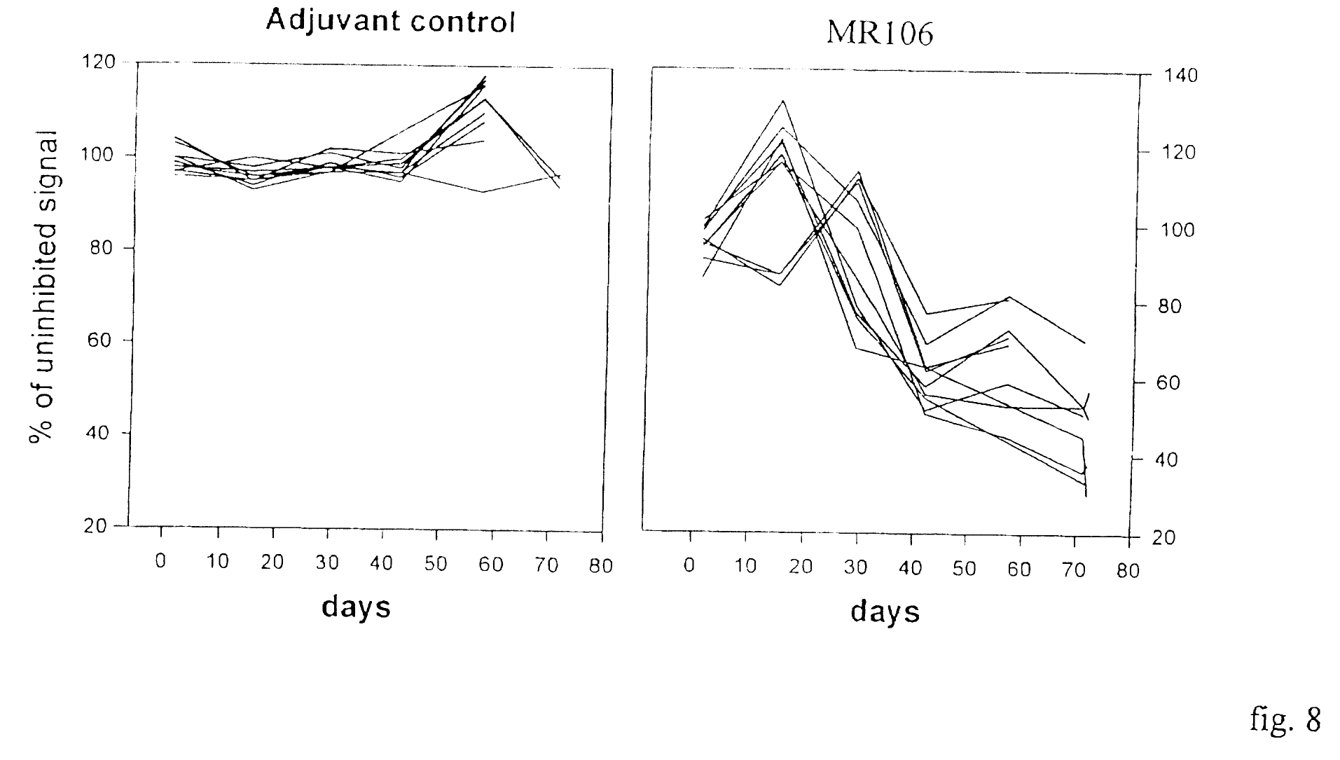

### Legend to FIG. 8

Measurement of ‘blocking antibodies’ in MR106 vaccinated and ‘adjuvant control’ lice. Each group comprised 5 C3H mice and 5 Balb/c mice. All sera tested were diluted 1:5. The inhibition was measured relative to a standard panel of ‘normal mice’ sera

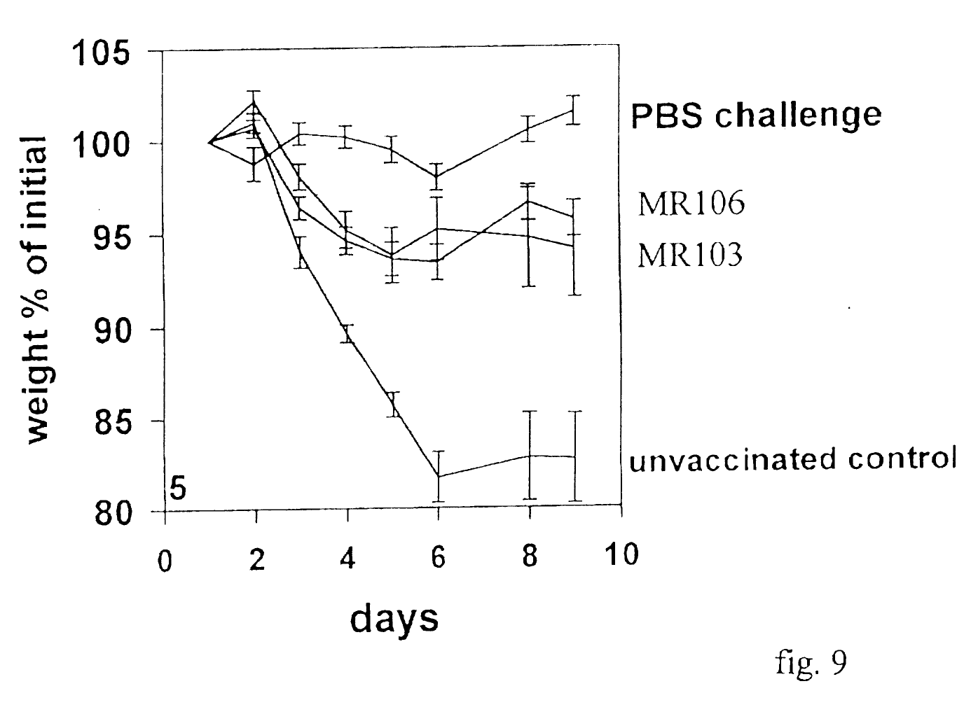

### Legend of FIG. 9

Challenge of C3H mice by daily injection of 20 μg purified TNFα in 200 μl. The weight was recorded in % of initial weight at the start of the challenge. The PBS challenge group consisted of 5 MR103-vaccinated, 5 MR106-vaccinated and 5 “adjuvant only” mice. MR106 (15 mice), MR103 (17 mice) showed a less severe weight loss compared with the control group A second control was challenged daily with 200 μl physiological PBS showed no weight loss The average weight was calculated for animals surviving at each time point

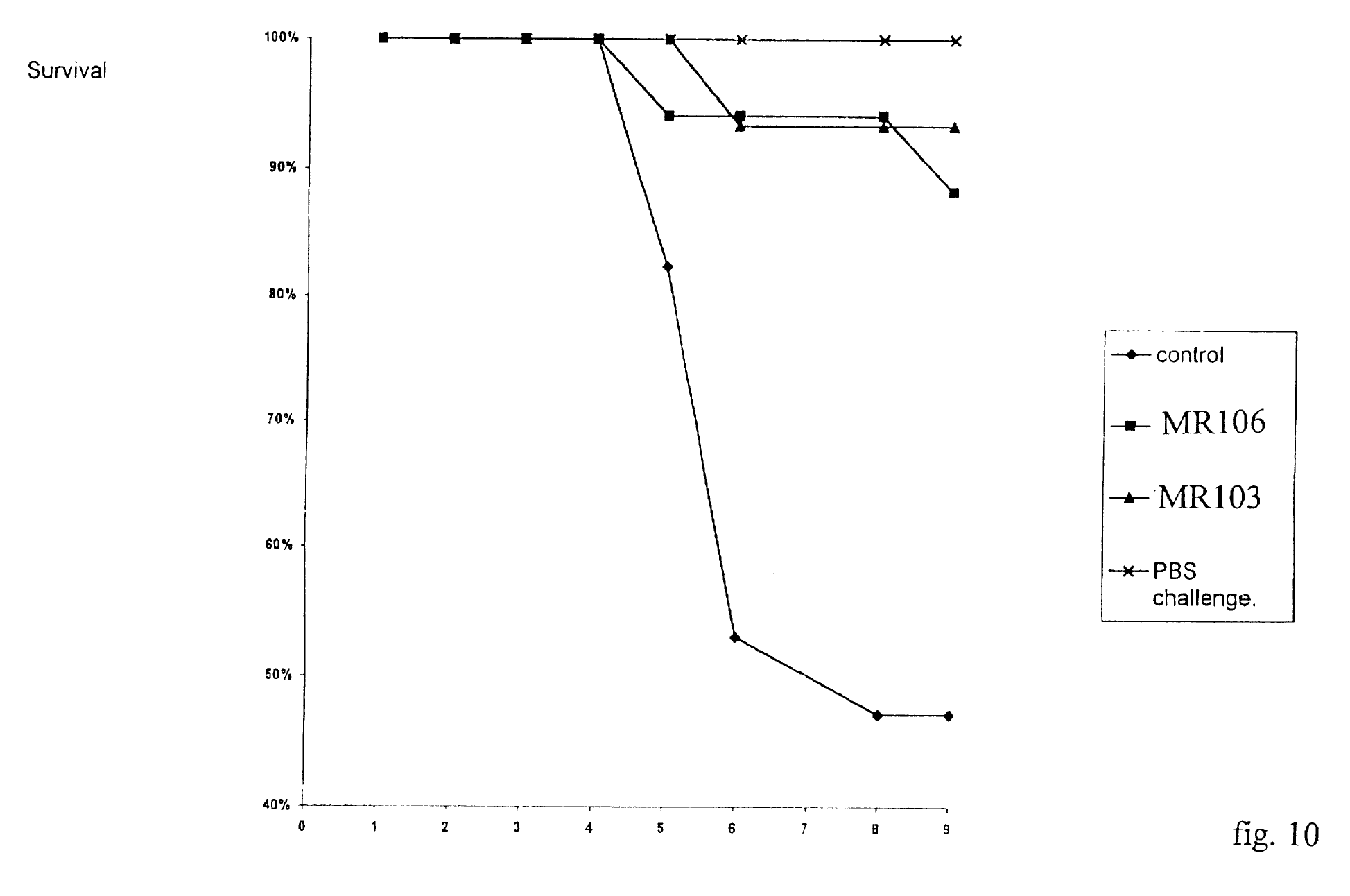

### Legend to FIG. 10

The survival curve during experimental cachexia. The same experiment as shown in FIG. 9 illustrating the survival rate in the 4 groups during the 9 day observation period. The massive lethality in the control group compared to the MR106 and MR103 vaccinated animals is evident

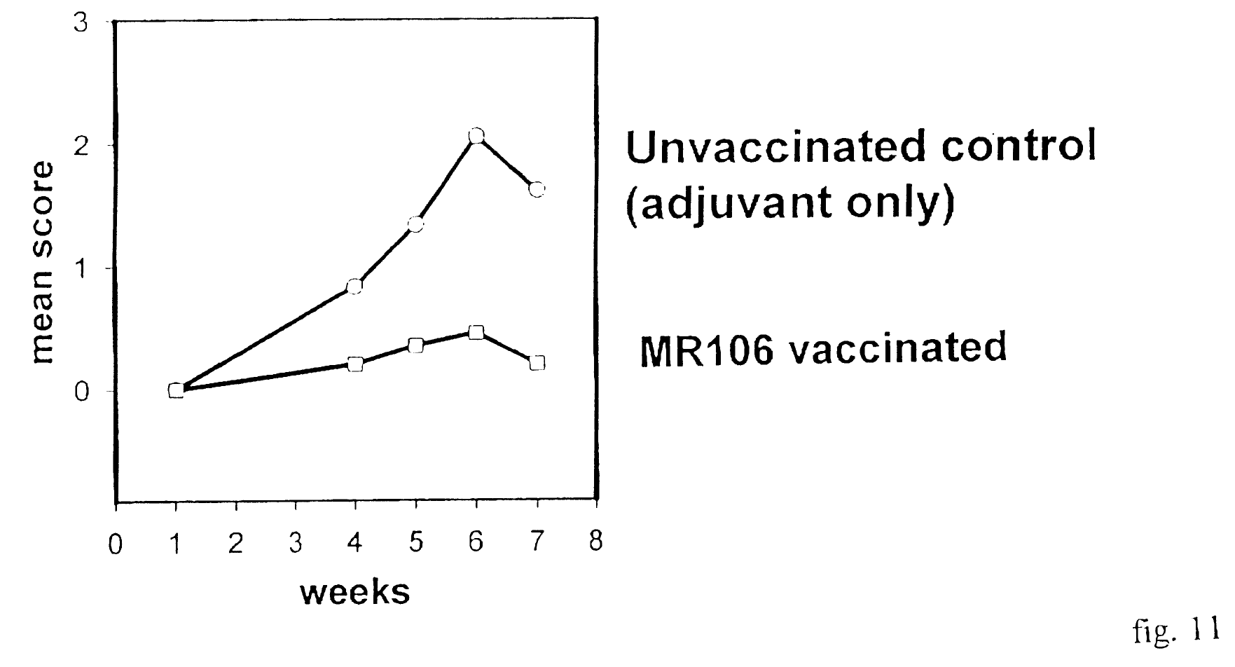

### Legend to FIG. 11

Collagen arthritis was induced by injection of two doses of 200 μg collagen type II. The TNFα vaccinated group received three doses of MR106 in FCA adjuvant (1. dose and incomplete adjuvant (subsequent doses). The arthritis developed after approximately 80 days and was recorded for a total of 8 weeks by a blinded observer.

### DESCRIPTION OF THE PREFERRED EMBODIMENTS

The present invention is based on the surprising fact that injection of recombinant self-proteins, which have been appropriately modulated by deletion of one ore more peptide fragments and simultaneous insertion of a corresponding number of foreign T cell epitopes, so as to produce a self protein analog with an essentially preserved tertiary structure induces a profound autoantibody response against the unmodified self-proteins.

By inducing minimal tertiary structural changes in the highly conserved self-protein, ubiquitin (Example 1) as well as in autologous TNFα (Example 3), foreign T cell epitodes having a length of 12-15 amino acids have been inserted using genetic engineering methods. These recombinant self proteinanalogs were purified emulsified in adjuvant and injected into mice. Within only one week autoantibodies against ubiquitin could be detected in serum from these mice (Example 2). Autoantibodies against native, autologous TNFα could be detected within a comparable time period (Example 3). Native ubiquitin or TNFα was not able to induce a autoantibody response.

By using the herein described principle for developing vaccines against undesirable proteins, the risk of inducing allergic side-effect is reduced and toxic self-proteins such as TNFα can simultaneously be de-toxified by removing or mutating biologically active protein segments. The epitope-shielding, effect described above is not a problem, and the autoantibodies were induced much faster as compared to the known technique, in which the self-protein is conjugated to a carrier protein or peptide (Example 4). Furthermore, by insertion of the T-cell epitopes at different positions the fine specificity of the autoantibodies can be regulated, potentially enabling a turning of the specificity towards a specificity mediating, high neutralizing effect on the desired biological activity (Example (6). This is an important practical feature of the present invention compared to all other methods previously published.

Importantly, recombinant proteins modified according to the method furthermore are self-immunogenic in large population expressing different MHC class II molecules. Surprising it was thus shown that the MHC-restriction of the autoantibody response induced was not necessarily confined to that of the inserted T cell epitope. Modulating of autologous ubiquitin and TNFα according to the present invention wherein the self-protein analog is produced by substitution of one or more peptide fragments by a corresponding number of peptides known to contain immunodominant T-cell epitopes, said substitution being carried out so as to essentially preserve the overall tertiary structure of the original self-protein, it was possible to induce an equally fast and even stronger autoantibody response against TNFα despite the fact that the inserted T cell epitope used was not restricted to the MHC molecules of the immunized mice (Example 2, 3 and 4). The reason for this observation is not clear but may be due to the appearance of new MHC binding segments of the mutagenized area in the self-protein. However, the experiment shown in Example 5 below demonstrates that this may not be the case, since synthetic peptides representing overlapping regions of the implanted ovalbumin T cell epitope in ubiquitin apparently did not bind strongly to any of the MHC class II molecules of the H-2^{k} mice in which this recombinant molecule was highly immunogenic. The observed lacking correspondence between the MHC restriction of the inserted T cell epitope and the restriction of the antibody response could therefore perhaps also be due to a general disturbance of the intra-molecular competition of self-protein segments. According to the method of the invention (non-tolerized) cryptic self-protein segments may be presented to the T cells leading to breaking of the T cell as well as the B-cell autotolerance towards the protein.

In accordance with the invention as illustrated in all the examples described below, a fragment of the self-protein was substituted with a foreign T cell epitope. This deletion followed by a substitution with another protein fragment minimally obscure the tertiary structure of the self-proteins, but may still contribute strongly to the disturbance of said intra-molecular molecular competition of MHC class II binding self-segments. This concept is clearly different from the above mentioned prior art mechanisms and methods.

Independently of the mechanism of action by the method according to the invention, it is more technically advantageous compared to the previously known methods for breaking, the B-cell autotolerance, since it is possible to induce antibodies in a broad population of MHC molecules by insertion of a minimal number different foreign T cell epitopes.

Using the present method a murine vaccine against autologous TNFα was prepared. The antibodies raised in mice vaccinated with this was shown to interfere with the ability of murine TNFα to interact with the TNFα receptor (Example 7). Furthermore, it has also been demonstrated that vaccination of C3H mice as well as Balb/c mice protects them against TNFα induced cachexia and death (Example 8). Finally, it has been convincingly shown that vaccination of DBA/1 mice against TNFα was able to protect these against collagen type II induced arthritis (Example 9). In conclusion, from these data it is clear that the method according to the present invention can be used to induce a very effective autoantibody response against self-proteins including pathogenic self-proteins such as TNFα. This inflammatory cytokine is known to play an important role in chronic inflammatory diseases, most notably rheumatoid arthritis. Therefore the herein described method can be used for preparation of a therapeutic vaccine against e.g. this disease (Example 10).

The vaccine according to the invention consists of one or more self-protein analogs modulated as described above and formulated with suitable adjuvants, such as calcium phosphate, saponin, quil A or biodegradable polymers. The modulated self-protein analog may optionally be prepared as fusion proteins with suitable, immunologically active cytokines, such as GMCSF or interleukin 2.

The autovaccine may i.a. be a vaccine against TNFα or γ-interferon for the treatment of patients with cachexia, e.g. cancer patients, or a vaccine against IgE for the treatment of patients with allergy. Further it may be a vaccine against TNFα, TNFβ or interleukin 1 for the treatment of patients with chronic inflammatory diseases.

### EXAMPLE 1

### Substitution of Foreign T Cell Epitopes into Ubiquitin

An overview to this procedure is shown in FIG. 1 using the T cell epitope MP7 as example. The gene sequences representing MP7 (MP7.1-C and MP7.1-NC) were synthesized as two complementary oligonucleotides designed with appropriate restriction enzyme cloning sites. The amino acid sequence of MP47 is PELFEALQKLFKHAY, (Mouritsen et al., Scand.J.Immunol. 30 723-730, 1989). The oligonucleotides were synthesized using conventional, automatic solid phase oligonucleotide synthesis and purified using agarose gel electrophoresis using low melting agarose. The desired bands were cut out from the gels, and known quantities of oligonucleotides were mixed heated to 5° C. below their theoretical melting point (usually to approximately 65° C.) for 1-2 hours, and slowly cooled to 37 C. At this temperature the hybridized oligonucleotides were ligated to the vector fragments containing the flanking parts of the ubiquitin gene. The subsequent analysis of positive clones using restriction fragment analysis and DNA sequencing was done by conventional methods (“Molecular Cloning”, Eds.: T. Maniatis et al. 2 ed. CSH Laboratory Press, 1989).

### EXAMPLE 2

### Induction of Autoantibodies Against Ubiquitin by Vaccination with Modified Ubiquitin Analogs

Genes containing sequences encoding the foreign T cell epitopes, OVA(325-336) from ovalbumin and HEL(50-61) from hen egg Ivsozyme respectively, were expressed in E. coli, AR58 under control of the heat sensitive λ repressor regulated promotor. Expression of the recombinant ubiquitin proteins were verified using a polyclonal anti-ubiquitin antibody and Western-blotting, (“Antibodies”, Eds.: D. Harlow et al., CSH Laboratory Press, 1988). The recombinant protein was purified using conventional methods (Maniatis et al., supra).

Mice were injected i.p. with 100 μg of ubiquitin or its analogs in phosphate buffered saline (PBS) emulsified in Freunds Complete adjuvant. Booster injections of the same amount of antigen emulsified 1:1 in Freunds Incomplete adjuvant were performed i.p. at days 14 and 28. Five Balb/c mice in each group were examined and blood samples were examined for the presence of anti-ubiquitin antibodies on day 7, 14, 21, 2, 35, and 42 using conventional ELISHA methodology.

The results exemplified by the antibody response against two different ubiquitin analogs containing the T cell epitopes OVA(325-336) and HEL(50-61) respectively, are shown in FIG. 2 The inserted amino acid sequence QAVHAAHEINE, OVA(325-336), and the inserted amino acid sequence STDYGILQINSR, HEL(5(0-6 1), contains the epitopes.

A clear antibody response against native ubiquitin could be detected within one week from the first injection of antigen reaching a maximum within 2 weeks. Anti-ubiquitin antibodies produced in rabbits by covalently conjugating ubiquitin to bovine immunoglobulin reached maximum values after a much longer immunization period (data not shown).

### EXAMPLE 3

### Induction of Autoantibodies Against Tumor Necrosis Factors (TNFα) by Vaccination with Appropriately Modified TNFα Analogs

The gene coding for the structural part of the native murine TNFα protein (MR101) was obtained Polymerase Chain Reaction (PCR) cloning of the DNA. In the MR103 TNFα analog the ovalbumin (OVA) sequence #325-333-T (QAVHAAHAET), containing the T cell epitope, replaces the amino acids #26-35 in the cloned TNFα sequence, a substitution of anamphiphatic α helix. Substitutions in this region of the TNFα detoxifies the recombinant protein, (Van Ostade et al Nature 361, 266-269, 1993). In the MR105 TNFα analog the H-2² restricted T cell epitope from Hen Eggwhite Lysozyre (HEL) is contained in the amino acid sequence #81-96 (SALLSSDITASNCAK), which replaces the amino acids #5-20 in the cloned TNFα sequence. In the MR106 TNFα mutant the amino acid sequence (SALLSSDITASVNCA) HEL#81-95 containing the same T cell epitope, replaces the amino acids #126-140 in the cloned TNFα sequence. The genetic constructions are shown In FIG. 3, different techniques compared to that described in example 1is used, for exchanging parts of the TNFα gene with DNA coding for T cell epitopes. The MR105 and 106 constructs were made by introducing the mutant sequence by PCR recloning a part of the TNFα gene flanking the intended site for introducing the T cell epitope, the mutant oligonucleotide primer contained both DNA sequence homologous to the TNFα DNA sequence and DNA sequence encoding the T cell epitope. The PCR recloned part of the TNFα gene was subsequently cut with appropriate restriction enzymes and cloned into the MR101 gene. The MR103 construction was made by a modification of the “splicing by overlap extension” PCR technique (R. M. Horton et al Gene 77, 61, 1989). Here two PCR products are produced, each covering a part of the TNFα gene, additionally each PCR product contains half of the T cell epitope sequence. The complete mutant TNFα gene is subsequently made by combining the two PCR products in a second PCR. Finally the complete genetic constructions were inserted into protein expression vectors. Subsequently all genetic constructions were analyzed by restriction fragment analysis and DNA sequencing using conventional methods “Molecular Cloning”, Eds,: T. Maniatis et al 2.ed. CSH Laboratory Press, 1989). The recombinant proteins were expressed in E. coli and purified by conventional protein purification methods.

Groups of Balb/c (MHC haplotype H-2^{d}) and C3H (MHC haplotype H-2ₖ) mice, respectively, were immunized s.c with 100 μg of semipurified MR103 and MR106 emulsified in Freunds complete adjuvant. Every second week the immunizations were repeated using incomplete Freunds adjuvant. All mice developed an early and strong, antibody response against biologically active MR101. This was measured by a direct ELISA method using passively adsorbed pure MR101 (FIG. 4). Control mice immunized with MR101 and PBS. respectively, showed no antibody reactivity towards MR101.

Strikingly, the antibody response towards MR101 was not MHC restricted corresponding to the implanted T cell epitopes, since both mice strains of different MHC haplotypes responded well to MR103 and MR106 containing differently restricted T-cell epitopes (FIG. 4). Taken together these results illustrate, a) the ability of the self protein analogs, produced by the method according to the invention, to induce autoantibodies towards a secreted auto protein and, b) the improved efficiency of the herein described method with regard to inducing a response in a broader MHC population than predicted by the MHC binding ability of the inserted T cell epitopes. The immune response against MR101 induced by recombinant self-protein-analog MR103 and MR106 was stronger and much more high-titered compared to the immune response induced by aldehyde conjugated MR101 (Example 4).

### EXAMPLE 4

### Induction of Autoantibodies Against TNFα by Self Protein Analogs Produced by the Herein Described Method Compared to Unmodified Self-protein Conjugated to E. coli Carrier Proteins

The induction of autoantibodies against TNFα by the herein described method was directly compared to the autoantibody response induced when using a conjugate of TNFα and E. coli proteins which must contain small single T cell epitope peptides as well as larger foreign proteins.

Semipurified recombinant murine TNF (MR101) was conjugated to E. coli proteins in PBS, pH 7.4 using 0.5 formaldehyde. Conjugation of the proteins was confirmed by SDS-PAGE. These conjugates were subsequently used for immunization of C3H and Balb/c mice. Another group of mice was vaccinated with semipurified non-conjugated self protein analog MR105. About 100 μg of recombinant TNFα analog and conjugate were emulsified in Freunds complete adjuvant were injected subcutaneously in each group of mice. MR105 is biologically inactive as judged by the L929 cell assay. In subsequent immunizations every second week, incomplete Freunds adjuvant was used. Both groups eventually developed autoantibodies against highly purified biologically active MR101 as determined by ELISA, but the immune response against the non-conjugated analog MR105 produced by the method of the present invention was induced earlier and was of a higher titer (FIG. 5).

### EXAMPLE 5

### The Possible MHC Class II Binding of Peptides Representing Overlapping Sequences of Self-protein as well as an Inserted Ovalbumin T cell Epitope in Ubiquitin

Peptide-MHC complexes were obtained by incubating ¹²⁵I-labelled peptide (10-100 nM) with affinity purified MHC class II molecules (2- 10 μM) at room temperature for 3 days (S. Mouritsen, J Immunol. 148, 1438-1444, 1992). The following peptides were used as radiolabelled markers of binding: Hb(64-76)Y which binds strongly to the E^{k} molecule and HEL(46-61)Y which binds strongly to the A^{k} molecule. These complexes were incubated with large amounts of cold non radiolabelled peptide (>550 μm) which should be sufficient to inhibit totally all immunologically relevant MHC class II binding. Either the same peptides were used or were three different overlapping peptides representing the flanking regions as well as the entire OVA(325-336) sequence, containing the T cell epitope, which was substituted into ubiquitin (see Example 2). The three peptides were: TITLEPSQAVHAA (U(12-26)), PSQAVHAAHAEINEKE (U(19-34)) and HAEINEKEGIPPDQQ (U(27-41)). The reaction buffer contained 8 mM citrate, 17 nM phosphate, and 0.05% NP-40 (pH 5) and peptide-MHC class II complexes were separated (in duplicate) from free peptide by gel filtration using G25 spun columns. Both the radioaictivities of the excluded “void” volume and of the included “void” were measured by gamma spectrometry. The competitive inhibition of maximal binding (in percent) by addition of cold peptide was calculated. The results are shown in Table 1.

TABLE 1

| Peptid/ | | | | | |
|---|---|---|---|---|---|
| MHC | Hb(64-76) | HEL(46-61) | U(12-26) | U(19-34) | U(27-41) |
| A^{λ} | 28.6 | 97.4 | 35.3 | 44.6 | 7.8 |
| E^{λ} | 92.6 | 0.0 | 45.6 | 12.2 | 0.0 |

It can be seen that the total inhibition binding of the radiolabelled peptides Hb(64-76)Y and HEL(46-61)Y to E^{k} and A^{k} respectively could only be achieved using the same cold versions of the peptides, Although some inhibition of binding was seen by U(12-26) and U(19-34) using these extreme amounts of cold peptide, it is likely that the affinity of these peptides to the H-2^{k} class II molecules is very low. Therefore this seems not to be sufficient to explain the strong immunogenicity of in the H-2^{k} mouse strain of the ubiquitin analog containing the ovalbumin T cell epitope. More likely, other and non-tolerized self-epitopes are presented to the T cells in these animals.

### EXAMPLE 6

### Difference in the Fine Specificity of Antibodies Raised Towards Different Ubiquitin Analogs

The fine specificity of the high titer antibodies raised in Balb/c mice towards recombinant ubiquitin containing OVA(325-336) and against recombinant ubiquitin containing HEL(50-61) (Example 2) was analyzed, and compared to the fine specificity of antibodies raised in rabbits towards denatured bovine ubiquitin which was chemically coupled to human IgG acting as a traditional carrier molecule.

Synthetic peptides corresponding to the following overlapping ubiquitin amino acid sequences: 1-15, 11-25, 21-36, 32-46, 42-56, 52-66, and 62-76 were covalently attached to activated microtiter plates (K. Gregorius et al, J. Immunol. Methods, 181, 65-73, 1995). In an ELISA assay antisera were added to the wells coated with one of the above mentioned peptides. Antibodies which bound to the peptides were subsequently detected with secondary antibodies coupled to alkaline phosphatase, which catalyses a chromogenic substrate reaction.

The results are shown in FIG. 6. The antibodies raised in response to recombinant ubiquitin containing OVA(325-336) reacted strongly with ubiquitin peptides 32-46 and 42-56, whereas the antibodies raised in response to recombinant ubiquitin containing HEL(50-6 1) were mainly directed towards ubiquitin peptides 1-15 and 32-46. in comparison, the antibodies raised towards the carrier-coupled bovine ubiquitin only reacted with the C-terminal ubiquitin peptide 62-76.

This result clearly shows that differently modified recombinant ubiquitin molecules elicit completely different anti-ubiquitin specificities. The hereby exemplified possibility of tuning the antibody response towards a desired fine specificity (e.g. towards a specificity mediating high neutralizing effect on biological activity) by using different insertion sites and/or different foreign epitopes in the modified self-proteins is a very importantadvantag,e of the present invention.

### EXAMPLE 7

Immunisation of Balb/c and C31H Mice Result in Autoantibodies Against TNFα Which Block TNFα /TNF-RECEPTOR 1 Interaction

Ten mice (5 Balb/c and 5 C3H mice) were immunised with 5 doses of MR106 (see example 3) during a period of 72 days. Freunds complete adjuvant was used for the first vaccination and Freunds incomplete adjuvant for all subsequent immunisations. An equivalent group designated “adjuvant control”, was vaccinated with physiological PBS in the same adjuvants. Antibodies against murine TNFα was produced by the MR106 vaccinated mice during the observation period These antibodies were able to block interaction between TNFα and TNFα-R1 (human TNFα Receptor 1). The amount of blocking antibodies was measured by an ELISA as illustrated and explained in FIG. 7. FIG. 8 illustrates how MR103 vaccinated mice gradually developed blocking antibodies (right panel) whereas the control immunised mice did not. This result clearly indicates that the autoantibodies has such a concentration, specificity and avidity that it is possible to interfere with the TNFα/TNF-R1 interaction..

### EXAMPLE 8

### Autoantibodies Against TNFα Protects Against Experimental Cachexia

C3H mice were immunized with four doses of MR103 or MR106 (see ekxample 3). Freunds complete adjuvant was used as adjuvant for the first immunisation. Incomplete adjuvant was used for all subsequent immunisations. Control mice were treated with the same adjuvant but active ingredients were replaced with physiological PBS. Three groups of mice (MR106:15 mice, MR103:17 mice, and ‘adjuvant only’ 17 mice were challenged by daily injections of 20 82 g of purified murine TNFα . The results are shown in FIG. 9). The ‘adjuvant only’ group developed a very significant weight loss of up to 20 % of body weight, whereas the MR106 and MR103 vaccinated animals developed only a small weight loss. A control group consisting of 5 MR103-vaccinated, 5 MR106-vaccinated, and 5 ‘adjuvant only’ mice received daily i.p injections with physiological PBS. These mice developed no weight loss. The relative was calculated using the entry weight of each animal as reference. The average relative weight was calculated based on surviving animals at each time point. A survival curve Of the same animals is illustrated in FIG. 10. An identical experiment performed using Balb/c mice gave equivalent results. These experiments show that autoantibodies to TFNα can be induced by TNFα mouse strains of various MHC haplotypes. These antibodies Can neutralise an otherwise lethal and cachectic dose of exogenously administered TNFα.

### EXAMPLE 9

### Autoantibodies Against TNFα Protect DBA1 Mice Against Collagen Induced Arthritis

Eighteen DBA/1 mice (MHC-haplotype H2^{q}) were vaccinated with three doses of MR106 at week 0, week 2 and week 4. Furthermore 200 μg of collagen type II was injected s.c. on week 0 and week 3. A corresponding control group of 18 mice were vaccinated with physiological PBS and collagen type II. 80 days after the first vaccination control mice started developing typical signs of collagen induced arthritis. At this time point the arthritis of each paw was classified as mild (score 1), significant (score 2) or severe (score 3) by a blinded observer. The mean score in each group is illustrated in FIG. 11. The MR106 vaccinated mice developed only mild symptoms of arthritis during the observation period, compared with the control group Wien the arthritis reached the peak value the number of affected animals (animals with one paw scoring 1 above) in the control group was significantly higher than in the MR106 vaccinated group (p<0.03) This experiment clearly shows the beneficial effect of neutralising TNFα with autoantibodies in murine collagen induced arthritis.

### EXAMPLE 10

### Treatment of Diabetes or Inflammatory Disease by Vaccination with Appropriately Modified TNFα Analogs

Genes encoding, human TNFα are modified by substitution at appropriate positions with one or more appropriate gene segments coding for T cell epitopes derived from e.g. tetanus toxin or influenza hemaglutinin. Such genes are expressed in appropriate expression vectors in e.(, E. coli or insect cells. The recombinant TNFα proteins is purified using conventional methods (“Molecular Cloning”, Eds. T. Maniatis et al. 2. ed. CSH Laboratory Press, 1989). Optionally such recombinant proteins can be coupled to immunologically active cytokines such a GM-CSF or interleukin 2 to further enhance the immunogenicity of the constructs.

The recombinant proteins can be formulated with appropriate adjuvants and administered as an anti-TNFα vaccine to patients suffering from diseases where TNFα is important for the pathogenesis. The induced anti-TNFα antibodies will thereby ameliorate the diseases.

One example of said diseases is the chronic inflammatory diseases such as e.g. rheumatoid arthritis where TNFα is believed to play an important role (reviewed in: F. M. Brennan et al. Br. J. Rheumatol. 31, 293-298, 1992). TNFα is also believed to play an important role in the cachetic conditions seen in cancer and in chronic infectious diseases such as AIDS (reviewed in M. Odeh, J, Intern. Med. 228, 549-556, 1990). It is also known that TNF participate in septic shock (reviewed in: B. P. Giroir. Crit. Care. Med., 21, 780-789, 1993). Furthermore, it has been shown that TNFα man play a pathogenetic role in the development of type II diabetes mellitus (CH Lan et al., Endocrinology, 130, 43-52, 1992).

1. A method for the modulation of self-proteins so as to induce antibody response against such proteins following administration of said modulated self-proteins in the host of said self-proteins,
which comprises providing a self-protein analog by molecular biological means by substitution of one or more peptide fragments of the self-protein by a corresponding number of peptides known to contain immunodominant foreign T-cell epitopes, said substitution being carried out so as to essentially preserve the overall tertiary structure of the original self-protein,

2. The method according to claim 1, wherein said immunodominant foreign T-cell epitope is inserted so as to preserve flanking regions from the original self-protein comprising at least 4 amino acids on either sides.

3. The method according to claim 1 wherein said T-cell epitope(s) comprise(s) at least 10 amino acids

4.The method according to claim 3, wherein said T-cell epitope(s) comprise(s) at least 15 amino acids.

5. The method according to of claims 1, wherein the immunodominant T-cell epitope(s) originate(s) from tetanus toxoid or diphtheria toxoid.

6. An autovaccine against undesirable self-proteins in humans or animals, which comprises one or more self-proteins analogs modulated according to any of claims 1-4 and formulated with pharmaceutically acceptable adjuvants, such as calcium phosphate, saponin, quil A or biodegradable polymers.

7. The autovaccine according to claim 6, wherein the self-protein analog is present in the form of a fusion protein with suitable, immunologically active cytokines, such as GM-CSF or interleukin 2.

8. An autovaccine according to claim 6, which is a vaccine against TNFα or γ-interferon for the treatment of patients susceptible to cachexia, e.g. cancer patients.

9. An autovaccine according to claim 6, which is a vaccine against IgE for the treatment of it patients with allergy.

10. An autovaccine according to claim 6, which is a vaccine against TNFα, TNFβ or interleukin 1 for the treatment patients with chronic inflammatory diseases.

11. An autovaccine according to claim 10, which is a vaccine for treatment of patients with rheumatoid arthritis or inflammatory bowel disease.

12. An autovaccine according to claim 6 and 7 which is a vaccine against TNFα for the treatment of diabetes mellitus.
